(19) United States
(12) Patent Application Publication
(10) Pub. No.: US 2006/0076237 A1
(43) Pub. Date: Apr. 13, 2006

(54) Coated hydrophilic membrances for electrophoresis applications

(75) Inventor: Malcolm G Pluskal, Acton, MA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: PROTEOME SYSTEMS INTELLECTUAL

(21) Appl. No.: 10/518,328

(22) PCT Filed: Jun. 16, 2003
(86) PCT No.: PCT/AU03/00750

§ 371 Date: Sep. 8, 2005

(30) Foreign Application Priority Data
Jun. 17, 2002 (AU) PS2957

(51) Int. Cl. C07K 1/26; G01N 27/447

(52) U.S. Cl. 204/450; 204/600

(57) ABSTRACT

The present invention is directed to an electrophoresis gel plate for analysing or separating macromolecules in a mixture comprising a polymerised gel matrix supported by a hydrophilic microporous membrane.

### FIELD OF THE INVENTION

The present invention relates to improved gel membranes that can be used in an apparatus for sub-fractionation, immobilised pH gradient gels or in general electrophoresis.

### BACKGROUND OF THE INVENTION

### DESCRIPTION OF THE RELATED ART

Gel electrophoresis is a well known technique for separating and analysing mixtures of macromolecules. Multicompartment electrolysers (MCE) were introduced in the late 1980's by Righetti P. G and co-workers (see U.S. Pat. No. 5,834,272) for processing large volumes and amounts of proteins to homogeneity.

A multi-compartment electrolyser can be used to pre-fractionate complex protein mixtures prior to separation by gel electrophoresis. Such a sub-fractionation process can effectively remove abundant macromolecules such as proteins present in large excess over other molecules in a cell lysate or body fluid. The fractioned protein mixture obtained is significantly devoid of such abundant components, and can be loaded in a separating gel at much higher levels, thus ensuring a greater sensitivity and detection capability of low-abundance proteins. An MCE can thereby produce protein fractions that are fully compatible with the subsequent gel electrophoresis protocols, since it is based on a charge based isoelectric focusing technique, which yields samples highly concentrated and low in salts and buffers.

Conventionally, a multi-compartment electrolyser comprises a stack of chambers sandwiched between an anodic and cathodic reservoir. The chambers are divided by isoelectric membranes, which comprise an acrylamide matrix incorporating one or more acrylamido buffers to provide the desired pI value and required buffering power.

International patent application No PCT/AU00/01391, filed in the name of Proteome Systems Limited and incorporated herein by reference, relates to such an electrolyser and to a method of using that electrolyser for sub-fractionation and subsequent separation of fractions from highly complex protein/peptide mixtures, such as those found in total cell lysates, body fluids and tissue extracts in general.

Hydrogel coated membranes used in an MCE are currently produced immediately prior to use. Membranes are produced by polymerisation of a mixture of acrylamido buffers and acrylamide monomers onto glass fibre membranes forming a hick (2-3 mm)-hydrogel layer having a relatively large volume. Unfortunately, this large volume can lead to adsorptive losses of proteins in the MCE separation process.

Further, the casting technique is relatively difficult due to the possible formation of bubbles and irregularities in the gel. When cast, the hydrogel coated membranes are fragile and difficult to handle. As the gels are commonly cast in a high concentration of urea, they need to be stored at room temperature, do not have a long storage life, and cannot be air-dried for long term storage without the collapse of the hydrogel layer.

In work leading up to the present invention, the inventor sought to develop an improved gel membrane or gel plate that is stable, relatively easy to handle, can be dried and stored in a desiccated state. The inventor particularly sought to develop improved membrane supported gels suitable for use in a multi-compartment electrolyser, in isoelectric focussing, or for general electrophoresis applications.

General Information

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment and, in particular, with respect to the gel plate and its use in electrophoresis shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally-equivalent products, compositions and methods are clearly within the scope of the invention, as described herein.

The present invention is performed without undue experimentation using, unless otherwise indicated, conventional techniques of molecular biology, electrophoresis, and gel technology. Such procedures are described, for example, in the following texts that are incorporated by reference:

Sambrook, Fritsch & Maniatis, Molecular Cloning: A Laboratory Manual, Cold Spring Harbor Laboratories, New York, Second Edition (1989), whole of Vols I, II, and III.

### SUMMARY OF INVENTION

The present invention provides electrophoresis gel plates for separating and/or analysing macromolecules in a mixture comprising a polymerised gel matrix supported by a hydrophilic microporous membrane.

Conventionally, porous membrane filters are formed from a solid polymeric matrix and are adapted to be inserted within a fluid stream to effect removal of particles, microorgansims or a solute from liquids and gases. The inventor has surprisingly found that hydrophilic microporous membranes can be used to support a polymerised gel matrix to form a gel plate. Advantageously, the hydrophilic microporous membranes do not substantially inhibit polymerisation or cross-linking of gel forming monomers and polymers.

Accordingly, in a first aspect the present invention is directed to an electrophoresis gel plate for analysing or separating macromolecules in a mixture comprising a polymerised gel matrix supported by a hydrophilic microporous membrane.

In one embodiment the improved gel plate is dried, preferably substantially without the collapse or physical damage of the gel matrix. Preferably, the dried gel plates of the present invention can be stored in a desiccated state.

The term “support” or “supported” refers to a close physical relationship between the gel matrix and the hydrophilic microporous membrane or juxtaposition or contacting of these integers. Preferably, the gel matrix binds to or adheres to the hydrophilic microporous membrane. Preferably, the gel matrix is adsorbed by the hydrophilic microporous membrane.

Preferably, the improved gel plate shows good adhesion of the gel matrix to the hydrophilic microporous membrane surface, and good mechanical properties to stabilise the dimensions of a gel plate.

In one embodiment, the hydrophilic microporous membrane is a hydrophilic-coated microporous membrane.

Preferably, the electrophoresis gel plate is capable of being dried and is suitable for long-term storage.

The preparation of gel plates according to the present invention can be carried out with well known techniques. In one embodiment, the hydrophilic microporous membrane is wet with a casting solution and the casting solution is treated to effect polymerisation.

By “wet” is meant that the microporous membrane is contacted or applied or soaked or impregnated with the casting solution.

By “casting solution” is meant a solution of gel forming materials known to be suitable for electrophoresis purposes. Preferred materials include acrylamide, suitable buffers, and/or agarose.

Accordingly, in a second aspect, the present invention provides a process of preparing an electrophoresis gel plate according to the first aspect of the invention, the process comprising wetting a hydrophilic microporous substrate with a casting solution, and polymerising the casting solution to form a polymerised gel matrix supported by the hydrophilic microporous substrate.

In a third aspect, the present invention provides use of an electrophoresis gel plate in the separation or analysis of at least one macromolecule in a mixture, wherein the electrophoresis gel plate comprises a polymerised gel matrix supported by a hydrophilic microporous substrate.

In a fourth aspect, the invention provides a method for separating or analysing macromolecules in a mixture comprising
(i) placing the mixture of macromolecules in a separation apparatus comprising at least one electrophoresis gel plate, and
(ii) performing electrophoresis,
   wherein the electrophoresis gel plate comprises a polymerised gel matrix supported by a hydrophilic microporous substrate.

In one embodiment, the separation apparatus is a multi-compartment electrolyser.

In a fifth aspect, the present invention provides a kit for analysing or separating macromolecules in a mixture, the kit comprising one or more electrophoresis gel plates according to the first aspect of the invention, buffers and optionally including instructions for use.

### BRIEF DESCRIPTION OF THE FIGURES

Specific embodiments of the invention will now be described by way of example only and with reference to the accompanying drawings in which:—

FIG. 1 is a schematic view of an electrophoresis gel plate embodying the present invention.

FIG. 2 is a schematic exploded view of a multi-compartment electrolyser apparatus.

### DETAILED DESCRIPTION OF THE INVENTION

According to the present invention the hydrophilic microporous membrane supports a polymerised gel matrix to form a gel plate.

Gel Matrix

In one embodiment, the polymerised gel matrix comprises a cross-linked polyacrylamide gel. Polyacrylamide gels are choice media for electrophoresis because they are chemically inert and readily formed by the polymerization of acrylamide monomers. Pore sizes can be controlled by choosing various concentrations of acrylamide and a cross-linking reagent at the time of polymerization.

Methods for making or casting gels are well known in the art. Conventionally, acrylamide gel matrix compositions are described as % T/% C, wherein T is the total acrylamide and C is the amount of crosslinking agent.

Preferably, the gel matrix comprises about 2.5-10.0% total acrylamide concentration at a cross-link density of 2-15%.

In one embodiment the cross-linking agent is selected from the group consisting of bis-acrylamide, diacroyl piperazine, DATD, N,N′-diallyl-tartardiamide or BAC, N,N′-bis(Acryloyl) cystamine or alternate cross-linking agent or mixture thereof Preferably the crosslinker is bis-acrylamide.

Preferably, the gel matrix comprises about 2.5-8% total acrylamide concentration. More preferably, the gel matrix comprises about 2.5-7% total acrylamide concentration, more preferably about 2.5-6% total acrylamide concentration, more preferably about 3-5% total acrylamide concentration. Most preferably, the gel matrix comprises about 4% total acrylamide concentration.

Preferably, the cross-link density is about 4-15%. More preferably, the cross-link density is about 614%, more preferably about 7-13%, more preferably about 8-12%, more preferably about 9-11%. Most preferably the cross-link density is about 10%.

In a preferred embodiment the gel matrix comprises 4% T/10% C polyacrylamide solution. That is, the gel matrix comprises 4% total acrylamide of which 10% is from cross-linking bis-acrylamide.

In one embodiment, the gel matrix is a hydrogel. The term “hydrogel” herein refers to a three dimensional structure composed of cross-linked hydrophilic gel polymers, which are present in an expanded hydrated state in aqueous solution.

Preferably, the hydrogel is a cross-linked hydrogel.

Preferably, the gel matrix is an isoelectric focussing gel matrix. Preferably, the gel matrix is selected from the group consisting of a fixed pH isoelectric gel matrix, carrier ampholyte isoelectric gel matrix and immobilised pH gradient gel matrix.

Preferably, the gel matrix is a fixed pH isoelectric gel matrix. In one embodiment, the gel matrix comprises a polyacrylamide gel matrix comprising covalently attached buffers, preferably wherein the buffer has a defined pH. In one embodiment, the gel matrix comprises acrylamido buffers co-polymerised with cross-linked polyacrylamide.

In a particularly preferred embodiment, the gel matrix is a cross-linked hydrogel comprising acrylamide buffers.

In one embodiment the fixed pH isoelectric gel matrix has a pH value of between 2 and 12. The isoelectric point of a membrane can be adjusted to any value according to methods in the art. In one embodiment, the composition of the acrylamido buffers can be calculated to fix the pH of the matrix to a desired value.

Preferably, the pH of the gel matrix is selected from the group consisting of pH 3, 3.5, 4, 4.5, 5, 5.5, 6, 6.5, 7, 7.5, 8, 8.5, 9, 9.5, 10, 10.5, and 11.

Preferably, the pH isoelectric gel matrix is suitable for use in a multi-compartment electrolyser.

In an alternate embodiment, the gel matrix according to the present application comprises an isoelectric gel matrix having an immobilised pH gradient. In one embodiment, the immobilised pH gradient is in the range of 2 to 12. In alternate embodiments the immobilised pH gradient can be selected from a range of pH gradients including 2-10, 4-10, 5-9, 3-8, and 5-7.

Preferably, the immobilised pH gradient gel plates are suitable for use in two dimensional gel electrophoresis.

In another embodiment the gel matrix is a collapsed gel matrix, which can be reswollen in the presence of zwitterionic molecules (carrier ampholytes) to form a pH gradient on application of an electric field. This may be referred to as a carrier ampholyte based isoelectric gel matrix.

In one embodiment, the gel plate according to the present invention comprises a cross-linked polyacrylamide gel for general electrophoresis applications.

In an alternative embodiment of the invention, the gel matrix comprises agarose. Preferably agarose gels according to the present invention offer very large open pores sufficient for the passage of large molecules or organelles, such as, for example, mitochondria or nuclei.

In one embodiment, the gel matrix comprises about 0.5-2.0% (Wt/v) agarose. Preferably a gel matrix comprising agarose does not comprise a cross-linking agent.

In another embodiment, the gel matrix is a hybrid agarose-polyacrylamide gel.

In another embodiment of the invention, the thickness of gel matrices can be varied. The thickness can be altered by applying different volumes of casting solution to a hydrophilic membrane substrate positioned or held within a frame with spacers to contain the liquid to a required or desired depth over the membrane surface. On polymerisation, this process would lead to different thicknesses of the gel matrix.

In one embodiment of the present invention a thinner gel matrix is preferred- for reducing adsorptive loss in the gel matrix. In one application of the invention, the gel matrix thickness has a direct influence on the volume of protein loaded on gel plates.

Preferably, the gel matrix is about 10% (v/v) of the thickness of the microporous substrate. Preferably, the thickness is sufficient to substantially fill all the pores on the membrane and provide a layer of hydrogel on top of the membrane structure. In one embodiment, the gel matrix is between about 0.01 mm and about 5 mm thick. Preferably, the gel matrix is between about 0.05 mm and about 4 mm thick, more preferably between about 0.1 mm and about 3 mm, more preferably between about 0.05 mm and about 2 mm, more preferably between about 0.1 mm and about 1 mm, more preferably between about 0.15 mm and 0.5 mm.

In an alternate embodiment, the gel matrix is between about 0.01 mm and about 0.5 mm thick, more preferably, between about 0.02 mm and about 0.4 mm, more preferably between about 0.03 mm and about 0.3 mm, more preferably between about 0.04 mm and about 0.2 mm, more preferably between about 0.05 mm and about 0.1 mm. In one embodiment the gel matrix is a monolayer. Preferably, this is achieved by coating the membranes with minimal amount of polyacrylamide-acrylamido buffer matrix so as to leave the membrane porosity largely unchanged.

Hydrophilic Microporous Membrane

In one embodiment, the microporous membrane comprises a hydrophilic or partially hydrophilic membrane. Preferably, the nicroporous membrane is constructed of a polymeric material. In an alternate embodiment, the microporous membrane is not constructed of a polymeric material.

In a preferred embodiment, the microporous membrane is constructed of a polyamide, such as for example nylon. In an alternate embodiment the microporous membrane is constructed of a cellulosic material, such as cellulose, regenerated cellulose, cellulose acetate, or nitrocellulose.

In another embodiment, the hydrophilic microporous membrane is constructed of a mixture of polymeric materials.

In yet another embodiment, the hydrophilic microporous membrane comprises a porous substrate, and an insoluble cross-linked hydrophilic material. Preferably the insoluble cross-linked hydrophilic material coats the porous substrate. “Coating” or “coats” refer to a close physical relationship between the substrate and the hydrophilic material or juxtaposition or contacting of these integers. Preferably, the hydrophilic material binds to or adheres to the substrate. Preferably, the substrate is entirely coated or covered by the hydrophilic material. Preferably, the hydrophilic material is adsorbed by the substrate. Methods for modifying a porous substrate to provide a hydrophilic microporous membrane are known in the art. Methods of rendering a porous fluorocarbon resin hydrophilic are described for example in U.S. Pat. No. 4,113,912 (1978).

Preferably, the substrate is constructed of a polymer. More preferably, the polymer is a porous polymer. Preferably, the porous polymer forming the substrate is selected from the group consisting of fluorinated polymers such as poly(tetrafluoroethylene) (TEFLON™), polyvinylidene fluoride (PVDF), and the like; polyolefins such as polyethylene, ultra-high molecular weight polyethylene (UPE), polypropylene, polymethylpentene, and the like; polystyrene or substituted polystyrenes; polysulfones such as polysulfone, polyethersulfone, and the like; polyesters including polyethylene terephthalate, polybutylene terephthalate, and the like; polyacrylates and polycarbonates; and vinyl polymers such as polyvinyl chloride and polyacrylonitriles.

Copolymers can also be used, such as copolymers of butadiene and styrene, fluorinated ethylene-propylene copolymer, ethylene-chlorotrifluoroethylene copolymer, and the like.

Suitable insoluble, cross-linked materials are one or more hydrophilic polymers, such as, for example, hydroxy propyl acrylate, polyvinyl alcohol, polyethyl glycol, and polyether sulfone, and regenerated cellulose or mixtures thereof.

Accordingly, hydrophilic microporous membranes can be made by rendering a porous substrate hydrophilic by coating with a thin layer of one or more hydrophilic polymers.

In one embodiment the porous substrate is in sheet form.

In a preferred embodiment the porous substrate has a defined pore size.

Pore sizes of the substrate can be varied. Preferably, the substrates have pore sizes from 0.65 to 5.0 micron. It is understood that other pore sizes having greater and smaller dimensions can also be used.

In a particularly preferred embodiment, the porous substrate can be selected from a range of PVDF membrane substrates such as those from Millipore Corporation including: films DVPP (0.65 micron), BVPP (1.2 micron) and web supported film SVPP (5 micron) of a range of pore sizes.

Gel Plate

Preferably, according to the present invention the close physical relation between the gel matrix and the surface of the hydrophilic microporous membrane provides stability for the gel plate. Stability may also be conferred by some covalent grafting of the polymer layers.

In a preferred embodiment, the pores of the hydrophilic microporous membrane can be filled with a polymerised gel. In one embodiment, polymerised gel is a cross-linked polyacrylamide gel. In one embodiment, the cross-linked polyacrylamide gel forms a continuous film. In one embodiment, the pores of the hydrophilic microporous membrane are filled such that the electrophoresis gel plate does not substantially allow liquid flow through the gel plate by induced pressure or passive diffusion (ie. without the application of the electric field).

This property can be used to test the uniformity and integrity of the gel matrix.

To measure liquid flow through the gel plate, the gel plate is placed on the surface of afritted glass filter manifold and a vacuum is placed under the filter. A drop of water is then placed on top of the filter and a vacuum applied. The rate and amount of water droplet flow through the gel plate is measured to determine liquid flow.

Preferably, a continuous film provides a particularly stable form of the gel plate. The main advantage of continuous film is that in use, liquid cannot flow through the gel plate without the application of an electric field. Hence, the gel plate can be used to isolate two fluid containing chambers.

In an alternate embodiment, the pores of the hydrophilic microporous membrane are not filled. In one embodiment, the cross-linked polyacrylamide gel forms a non-continuous film. In another embodiment, the cross-linked polyacrylamide gel partially fills the pores of the microporous substrate. The term “partially” is herein understood to mean that the gel plate retains some porosity. Porosity is retained if, for example, not enough gel solution is provided to fill all the hydroplilic membrane pores. In this case, when vacuum is applied the water droplet will rapidly flow through the gel plate.

Preferably, a non-continuous film provides a stable gel plate and has open porosity. In one embodiment, the gel plate comprising a non-continuous film is useful for separations with larger structures such as organelles or whole cells.

In a particularly preferred embodiment, the electrophoresis gel plate is stable, can be dried and is suitable for long term storage.

According to the present invention, a stable gel plate is one that can be washed dried and stored for a convenient amount of time before being used. Preferably, a stable gel plate is able to be stored for up to 1 year at room temperature or cool temperatures without loosing its functionality. Preferably, the gel plate is able to be rehydrated. Preferably, the rehydrated gel plate provides the established pH surface property, and more preferably the rehydrated gel plate shows relatively little tendency to become brittle or less pliable. Preferably the gel plate is substantially resistant to chemical breakdown of the polymerised hydrogel for time while it is stored in suitable conditions (dark room or cool temperatures).

Preferably, gel plates according to the present invention are tested for stability according to standard methods as described in
1) Kirkwood, T. B. L Predicting the stability of biological standards and products. Biometrics 33:736-742 (1977)
2) Porterfield, R. I, and Capone, J. J. Applications of Kinetic models and Arrhenius methods to product stability evaluations. Med. Devices Diagn. Industry April 1984, pg 45-50.
3) Kennon, L. Use of models in determining chemical pharmaceutical stability. J. Pharm. Sci. 53: 815-818 (1964)

In another aspect, the present invention is directed to an electrophoresis gel plate for analysing or separating macromolecules in a mixture.

Preferably analysing or separating including isoelectric focusing, native and SDS denatured size separation.

In one embodiment the electrophoresis gel plate suitable for use in an MCE.

Alternatively, the electrophoresis gel plate is an immobilised pH gradient gel strip suitable for use in isoelectric focussing. Or alternatively, the electrophoresis gel plate is a cross-linked polyacrylamide gel suitable for use in general electrophoresis applications.

The term “general electrophoresis applications” refers to the resolution of a complex mixture on the basis of charge on the species, and in addition, on the basis of molecular size and hence mass. These separation tools are used to resolve complex mixtures of analytes, such as proteins, nucleic acids and carbohydrates.

In one embodiment, the gel plate of the present invention is suitable for use as a gel for electrophoresis of biomolecules (eg proteinaceous molecules, including proteins, protein fragments, peptides, protein complexes) such that the gel has two-dimensional spatial stability and the support is substantially non-interfering with respect to detection of a label associated with one or more biomolecules in the gel (eg. a fluorescent label bound to one or more proteins).

In a second aspect, the present invention provides a process of preparing an electrophoresis gel plate according to the first aspect of the invention, the process comprising wetting a hydrophilic microporous substrate with a casting solution, and treating the casting solution to effect polymerisation to form a polymerised gel matrix supported by the hydrophilic microporous substrate.

Preferably, the process further comprises preparing a casting solution.

The process for making a gel plate is well known in the art. For example this process is clearly described in U.S. Pat. No. 5,928,792 (Millipore Corporation), which describes a process for producing a porous membrane product. Further descriptions of the preparation of gels suitable for electrophoresis are described in, for example, U.S. Pat. No. 4,243,507 (Martin et al).

Preferably, the casting solution comprises acrylamide/bis monomers and acrylamido buffers.

In an alternate embodiment the casting solution comprises agarose.

Preferably, after wetting the hydrophilic microporous membrane with the casting solution, the hydrophilic microporous membrane is allowed to adsorb the gel solution.

In one embodiment, after the hydrophilic microporous membrane is allowed to adsorb the casting solution, the hydrophilic microporous membrane and casting solution are subjected to a mechanical force to remove excess gel solution. In an alternate embodiment the hydrophilic microporous membrane and casting solution are not subjected to a mechanical force.

In an alternate embodiment, prior to the hydrophilic microporous membrane adsorbing the gel solution, the hydrophilic microporous membrane and gel solution are subjected to a mechanical force to remove excess gel solution.

In another embodiment, while the hydrophilic microporous membrane adsorbs the gel solution, the hydrophilic microporous membrane and gel solution are subjected to a mechanical force to remove excess gel solution.

In one embodiment the mechanical force is a roller.

In one preferred embodiment the roller is a wire wound roller.

In one embodiment, mechanical force can be suitably applied with a single roller contacted to one surface of the hydrophilic microporous membrane and casting solution. In one embodiment an air knife, a doctor knife, a scraper, an absorbent or the like is contacted with one surface of the hydrophilic microporous membrane and casting solution.

In an alternate embodiment mechanical force can be suitably applied with two rollers. Preferably, two rollers form a sandwich. Preferably if there are two rollers, the rollers allow the hydrophilic microporous membrane and casting solution to pass between the two rollers.

In one embodiment the casting solution is treated to effect polymerisation.

In another embodiment, the casting solution is treated in the presence of a catalyst. Preferably, the catalyst is TEMED.

In an alternate embodiment a catalyst is not present.

In yet another embodiment, the casting solution is treated in the presence of free radicals. Preferably, free radicals can be generated by a method well known in the art, such as, for example, decomposition of Ammonium persulfate, thermal decomposition of a suitable agent, light directed decomposition of a suitable agent (eg. riboflavin, methylene blue, or UV photocatalyst), or directly by short wavelength UV light, electron beam radiation, or ionization radiation (eg. gamma radiation).

Preferably, treating the casting solution comprises applying heat for a time and under sufficient conditions to effect polymerisation.

In an alternate embodiment, treating the casting solution comprises cooling for a time and under sufficient conditions to effect polymerisation.

In an alternate embodiment treating the casting solution comprises applying a sufficient amount of UV light to achieve polymerisation. Various wavelengths and times of exposure could be used to provide the right conditions. These conditions would be familiar or easily determined by a person skilled in the art.

In another embodiment, treating the casting solution comprises electron beam radiation for a time and under sufficient conditions to achieve polymerisation. Alternatively, those skilled in the art are aware of a sufficient amount of electron beam radiation (see for example U.S. Pat. Nos 4,704,198 and 4,985,128).

Preferably, the process further comprises recovering the gel plate comprising the polymerised gel matrix supported by the hydrophilic microporous substrate.

In one embodiment, the process further comprises washing and drying the gel plate.

It is also envisaged that this process can be “scaled up” for commercial purposes (see for example U.S. Pat. No. 5,271,839; Millipore Corporation). In a preferred embodiment a continuous thin film coating process is used. More preferably, conventional APS/TEMED catalysts can be used.

In a third aspect, the present invention provides use of an electrophoresis gel plate in the separation or analysis of at least one macromolecule in a mixture, wherein the electrophoresis gel plate comprises a polymerised gel matrix supported by a hydrophilic microporous substrate.

In one embodiment the electrophoresis gel plate is adapted for use in a multi-compartment electrolyser (MCE).

In an alternate embodiment, the electrophoresis gel plate is adapted for use in two-dimensional gel electrophoresis.

In a fourth aspect, the invention provides a method of analysing or separating macromolecules in a mixture the method comprising:
(i) placing the mixture of macromolecules in a separator apparatus comprising at least one electrophoresis gel plate, and
(ii) performing electrophoresis on the mixture
   wherein the electrophoresis gel plate comprises a polymerised gel matrix supported by a hydrophilic microporous membrane.

In one embodiment, the separator apparatus comprises electrodes for applying an electric field.

In one embodiment, the separation apparatus is a multi-compartment electrolyser.

Preferably, the multi-compartment electrolyser comprises two or more electrophoresis gel plates. Preferably, the two or more electrophoresis gel plates have different pI values.

In preferred embodiment, the multi-compartment electrolyser comprises three or more, preferably four or more, more preferably five or more electrophoresis gel plates. Preferably, the electrophoresis gel plates have different pI values. In a most preferred embodiment the electrophoresis gel plates are arranged such that the pI values increase monotonically from anode to cathode.

In a fifth aspect, the present invention provides a kit for analysing or separating macromolecules in a mixture, the kit comprising one or more electrophoresis gel plates according to the first aspect of the invention, buffers and optionally instructions for use.

In a preferred embodiment, the kit further comprises any one or more of the following:

urea, thiourea, CHAPS, carrier ampholytes and MCE apparatus.

In a preferred embodiment, the kit comprises two or more gel plats, more preferably three or more gel plats, more preferably four or more gel plates, more preferably five or more gel plats, more preferably six or more gel plates and so on.

Preferably, the kit comprises gel plates having different pH values.

By way of example, in one embodiment the kit comprises gel plates having a pH of pH3.0, gel plates having a pH of 4.6, gel plates having a pH of 5.4, gel plates having a pH of 6.2, gel plates having a pH of 7.0 and gel plates having a pH of 10.

In one embodiment of the third or fifth aspects, separated macromolecules can then be transferred by blotting to a suitable absorptive membrane, such as for example, PVDF hydrophobic membrane.

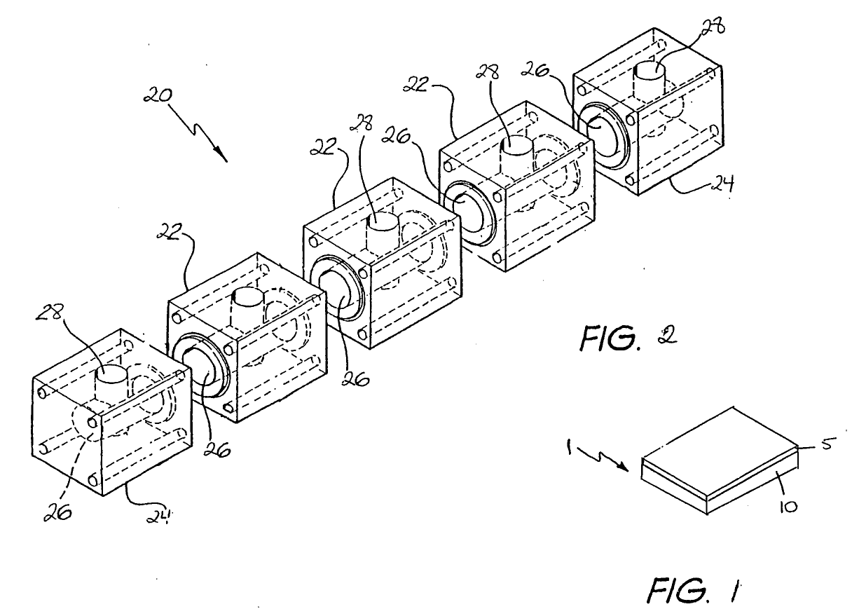

With reference to FIG. 1, the present invention provides an electrophoresis gel plate 1 for separating macromolecules comprising a polymerised gel matrix 5 supported by a hydrophilic microporous substrate 10.

In a particularly preferred embodiment, the electrophoresis gel plates of the present invention are suitable for use in a multi-compartment electrolyser (MCE). Preferably, proteins can only move between chambers by moving through the gel plate under electrophoresis conditions.

Referring to the drawings, FIG. 2 shows a disassembled separation apparatus in the form of a multi-compartment electrolyser apparatus 20. The apparatus includes five chamber blocks, defining three inner fractionation chamber blocks 22 and two, outer, electrode chambers blocks 24. In alternate embodiments the number of chambers can be varied as required. A cylindrical through bore 26 extends through the centre of each of the inner fractionation chamber blocks 22 and part way through the outer electrode chamber blocks 24. Each chamber block has a sample inlet 28.

With reference to FIG. 2, in the first operational mode (pre-fractionation) the multi-compartment electrolyser is assembled from a plurality of separate chambers, operating in an electric field, by placing dividers (not shown) between adjacent chambers. A divider comprises at least a gel plate having a known pI. In an MCE comprising a plurality of chambers and, therefore, a plurality of dividers, the gel plates have pI values increase monotonically from anode to cathode. The gel plates are sandwiched and seated so as to be flow-tight.

In the pre-fractionation mode, using one or more multi-compartment electrolysers, the device can be operated under denaturing conditions as customarily done in 2-D analysis, or alternatively, the device can be operated under native conditions, in the absence of denaturants, when native proteins are required for further analysis exploiting biological activity.

Accordingly, in use electrode solutions and sample solutions are added (and removed) via the sample inlets 28 in the top of each chamber. The sample inlets also allow excess fluid in a particular chamber to escape.

Proteins in the sample solution are driven through an isoelectric gel plate by the applied electric field which imparts mobility on charged proteins. The proteins contained therein will therefore migrate through the isoelectric gel plates towards the anode or cathode to reach the chamber in the MCE closest to the pI of the protein. Accordingly, the gel plates are able to trap a desired protein population within a given chamber.

Such a sub-fractionation process can effectively remove, via suitable narrow range isoelectric gel plates, proteins present in large excess in for example a cell lysate or in body fluids. In turn, the remaining protein mixture, devoid of such major components, can be loaded in a narrow pH range 2-D electrophoresis gel at much higher levels, thus ensuring a greater sensitivity and detection capability of low-abundance proteins.

Clearly, the multi-compartment electrolyser apparatus can have modified and improved features. Accordingly, a gel plate according to the present invention can be adapted in size and shape to fit various MCE apparatus.

### EXAMPLES

In order that the present invention may be more clearly understood preferred will be described with reference to the following non-limiting Examples.

Experimental

Membranes can be cast in a number of configurations; a) on the surface of a glass plate, or b) in a suitable vertical casting box using the Ammonium persulphate (APS)/TEMED catalysis process.

### Example 1

Casting MCE Gel Plates on Glass Plates

For production of pre-coated membranes a number of configurations are possible including a) on the surface of a glass plate, or b) in a suitable vertical casting box using the Ammonium persulphate (APS)/TEMED catalysis process.

Casting onto a glass plate is as follows;

Silane coat a glass plate (19×25 cm) with Rain X or a suitable silanizing agent to make glass surface water repellent.

Cut membrane to 16×22 cm.

Prepare 10 ml of the MCE membrane casting solution; and add catalysts

| Component | pH 3.0 | | pH 5.0 | | pH 8.0 | | pH 10.5 | |
|---|---|---|---|---|---|---|---|---|
| Acrylamido pK 3.1 | 0.500 | ml | | | 0.400 | ml | 0.066 | ml |
| Acrylamido pK 4.6 | | | 0.500 | ml | | | | |
| Acrylamido pK 8.5 | | | | | 0.500 | ml | | |
| Acrylamido pK 9.3 | 0.220 | ml | | | | | | |
| Acrylamido pK 10.3 | | | 0.380 | ml | | | 0.500 | ml |
| 30% T, 10% C | 1.33 | ml | 1.33 | ml | 1.33 | ml | 1.33 | ml |
| Acrylamide/bis | | | | | | | | |
| Acrylamide monomer | | | | | | | | |
| 1.0 M Tris base | 0.066 | ml | 0.026 | ml | | | | |
| 1.0 M Acetic acid | | | | | 0.018 | ml | 0.086 | ml |
| Urea | 2.4 | g | 2.4 | g | 2.4 | g | 2.4 | g |
| Water to final Volume | 5.0 | ml | 5.0 | ml | 5.0 | ml | 5.0 | ml |

Place solution in centre of glass plate and fold the membrane sheet and place the fold down into the casting solution. Allow the membrane to lie flat and absorb the casting solution for 2-3 min. Note: the solution should penetrate the microporous membrane to displace any trapped air. The membrane becomes translucent at this point. Cover the membrane surface with a suitable surface coated Mylar sheet (Gel Fix covers, Serva GmBh, Heidelberg, Germany) and express the excess casting solution with a roller.

Place another glass plate on top and heat to 50° C. for 1 h.

Remove upper glass plate and lift up the hydrogel coated membrane attached to the Mylar and then peel away the membrane and place into water for washing. Note: it is possible to see a thin clear layer of polymerised hydrogel on both sides of the microporous membrane. On contact with the water the urea diffuses out rapidly and the membrane reverts to its opaque appearance.

After 2-3 cycles of washing for 10 min each the membrane is placed in to a 2% (V/V) glycerol solution for 10 min prior to air drying supported in a frame to keep the membrane flat.

On air-drying the membranes are then stored at −20° C. in sealed storage bag until disks are punched out using a metal die.

### Example 2

To test and compare gel plates made according to the present invention, a sample of human plasma was fractionated on the MCE with each of the three types of membranes (hydrophilic PVDF with a 1.2 μm pore size, hydrophilic PVDF with 0.65 μm pore size and the SV membranes which are a web supported hydrophilic PVDF with a 5.0 μm pore size).

2-dimensional electrophoresis was carried out on the fractionated samples and protein yields and retention was determined using Bradford protein assays.

Preparation of Plasma

Plasma sample: Red Cross 2110475

8 mL of plasma containing 0.5% CHAPS was acetone precipitated at −20° C. for 30 minutes. The precipitate was recovered by centrifuging the sample at 5000 g for 10 minutes at 4° C. The pellet was resuspended in 80 mL of sample buffer (7M urea, 2M thiourea, 2% CHAPS and 5 mM tris. The sample was reduced with 5 mM TBP for 1 hour and reduced with acrylamide for 1 hour

MCE

The MCE was run with 5 chambers.

Anode chamber containing 5 mL of 7M urea, 2M thiourea and adjusted to pH 2.5 with orthophosphoric acid.

3.0-5.5 chamber, containing 5 mL of 7M urea, 2M thiourea and 2% CHAPS. 5.5-6.5 chamber, containing 5 mL of the plasma preparation.
(iv) 6.5-10.5 chamber, containing 5 mL of 7M urea, 2M thiourea and 2% CHAPS.
(v) Cathode chamber containing 5 mL of 7M urea, 2M thiourea and adjusted to pH 11.7 with 1M NaOH.

The pH values of the membranes were 3.0, 5.5, 6.5, and 10.3.

For each of the 3 runs, the unit was run at 100 volts for 4 hours and then at 1 watt for 20 hours.

Following fractionation, the volume of solution in each chamber was measured.

Protein concentration was determined using a modified Bradford assay. These results were used to determine concentration and dilution factors required.

Samples from the MCE chambers were either diluted or concentrated to 0.5 mg/mL prior to 2-D electrophoresis. The load sample was run at 1.5 mg/mL.

### Example 3

Isoelectric Focusing

180 μL of sample solution was used to rehydrate an 11 cm, pH 3-10 IPG strip. Prior to rehydration the sample had been coloured with orange G (0.01%) and centrifuged at 21000 g for 10 minutes at room temperature. The IPG strips were allowed to rehydrate for 6 hours.

The strips were focused using at 300 volts for 4 hours followed by a linear increase to 10 000 volts for 8 hours. The voltage was then maintained at 10000 volts until 50000 kvh or a current of less than 5 μA/gel was obtained.

Equilibration:

3.25 mL of equilibration buffer (50 mM tris-acetate, 6M urea, 2% SDS, 0.01% Bromophenol blue) was used to rehydrate the strips for 20 minutes.

### Example 4

SDS-PAGE

For SDS-PAGE GelChips (6-15% tris/acetate, lot number: P0264) were used. The gels were run at 50 mA/gel until the dye front had reached the bottom of the gel.

The gels were stained in coomassie G-250 for 18 hours with a change of stain after the first 2 hours. The gels were destained for 18 hours in 1% acetic acid.

### Example 5

Determination of Protein Retained in Membranes

The membranes were cut into small pieces (0.5 mm×0.5 mm) and transferred to a 2 mL eppendorf tube. A single tungsten carbide bead (3 mm) was added together with 1 mL of sample buffer. The tube was milled for 6 minutes at 30 hz. The tube was then centrifuges at 21000 g for 10 minutes and a protein assay carried out on the supernatant.

Results:

Protein Assays.

Table 1 shows the distribution of total protein in the MCE after fractionation.

The concentration of protein in the loaded sample was 5.14 mg/mL. Therefore, 25.7 mg of protein was loaded into the sample chamber. Table 2 shows the amount of protein retained on each of the MCE membranes after fractionation.

TABLE 1
Distribution of total protein after MCE fractionation.

| Membrane | | final | | |
|---|---|---|---|---|
| type | chamber | volume(mL) | mg protein | % protein |
| BV | 3.0-5.5 | 4.25 | 0.5 | 1.95 |
| BV | 5.5-6.5 | 5 | 20.5 | 79.72 |
| BV | 6.5-10.3 | 4.75 | 2.0 | 7.63 |
| | | | Total | 90.0 |
| | | | recovery | |
| DV | 3.0-5.5 | 4.25 | 0.3 | 1.17 |
| DV | 5.5-6.5 | 5 | 15.7 | 60.98 |
| DV | 6.5-10.3 | 4.5 | 2.0 | 7.86 |
| | | | Total | 70.4 |
| | | | recovery | |
| SV | 3.0-5.5 | 3.75 | 7.5 | 29.09 |
| SV | 5.5-6.5 | 6.5 | 20.6 | 80.20 |
| SV | 6.5-10.3 | 4.5 | 2.6 | 10.15 |
| | | | Total recovery | 120 |

TABLE 2
Protein retained on membranes after fractionation.

| | | | % of total |
|---|---|---|---|
| membrane type | pH | mg protein retained | protein retained |
| BV | 3.0 | <0.02 | <0.1 |
| BV | 5.5 | 0.10 | 0.4 |
| BV | 6.5 | 0.05 | 0.2 |
| BV | 10.3 | 0.03 | 0.1 |
| DV | 3.0 | <0.02 | <0.1 |
| DV | 5.5 | 0.04 | 0.2 |
| DV | 6.5 | 0.05 | 0.2 |
| DV | 10.3 | <0.02 | <0.1 |
| SV | 3.0 | 0.03 | 0.1 |
| SV | 5.5 | 0.21 | 0.8 |
| SV | 6.5 | 0.04 | 0.2 |
| SV | 10.3 | <0.02 | <0.1 |

2 Dimensional Analysis

The load sample was diluted to 1.5 mg/mL and focused on a pH 3-10, 11 cm EPG. Focusing was carried out using a linear increase in voltage from 300 to 10000 volts over 8 hours and then maintaining this voltage until 75kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

3.0-5.5 Fraction BV Type Membranes

The sample from the 3.0-5.5 MCE chamber was concentrated to 0.5 mg/mL and focused on a PSL pH 3-10 IPG. Focusing was carried out using a linear increase in voltage from 300 to 10000 volts over 8 hours and then maintaining this voltage until 75 kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

2 Dimensional Analysis of 5.5-6.5 MCE Chamber

The sample from the 5.5-6.5 MCE chamber was diluted to 0.5 mg/mL and focused on a PSL pH 3-10 IPG. Focusing was carried out using a linear increase in voltage from 300 to 10000 volts over 8 hours and then maintaining this voltage until 75 kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

2 Dimensional Analysis of 6.5-10.3 MCE Chamber

The sample from the 6.5-10.3 MCE chamber was diluted to 0.5 mg/mL and focused on a PSL pH 3-10 IPG. Focusing was carried out using a linear increase in voltage from 300 to 10000 volts over 8 hours and then maintaining this voltage until 75 kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

DV Type Membranes.

2 D gel of 3.0-5.5 (A), 5.5-6.5 (B) and 6.5-10.3 (C) chambers were run with DV type membranes. The samples were either diluted or concentrated to 0.5 mg/mL and focused on a PSL pH 3-10 IPG. Focusing was carried out using a linear increase in voltage from 300 to 10000 volts over 8 hours and then maintaining this voltage until 50 kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

SV Type Membranes

2 D gel of 3.0-5.5 (A), 5.5-6.5 (B) and 6.5-10.3 (C) chambers run with SV type membranes. The samples were either diluted or concentrated to 0.5 mg/mL and focused on a PSL pH 3-10 IPG. Focusing was carried out using a linear increase in voltage from 300 to 10000 volts over 8 hours and then maintaining this voltage until 50 kvh had been reached. SDS-PAGE was carried out using gel chips (6-15% gradient, lot number: P0264). Gels were run at 50 mA/gel for approximately 1 hour and 15 minutes. The gels were stained with Coomassie G-250 and destained with 1% acetic acid.

Discussion:

It is noted that good fractionation is achievable with the BV,DV and SV type membranes. The analysis that was carried out on the membranes suggest that the amounts of protein retained on the membranes are not significant. The maximum amount observed was 0.8% of the total protein retained on the SV 5.5 membrane.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An electrophoresis gel plate for analyzing or separating macromolecules in a mixture, the electrophoresis gel plate comprising a polymerized gel matrix supported by a hydrophilic microporous membrane.

2. The electrophoresis gel plate of claim 1, wherein the polymerized gel matrix comprises a cross-linked polyacrylamide gel.

3. The electrophoresis gel plate of claim 1, wherein the polymerized gel matrix comprises about 2.5-10.0% total acrylamide concentration.

4-17. (canceled)

18. The electrophoresis gel plate of claim 1, wherein the polymerized gel matrix is a hydrogel.

19. The electrophoresis gel plate of claim 1, wherein the polymerized gel matrix is an isoelectric focusing gel.

20. (canceled)

21. The electrophoresis gel plate of claim 19, wherein the polymerized gel matrix is a fixed pH isoelectric gel matrix.

22. The electrophoresis gel plate of claim 19, wherein the polymerized gel matrix comprises covalently attached buffers.

23. The electrophoresis gel plate of claim 19, wherein the polymerized gel matrix comprises acrylamido buffers co-polymerised with polyacrylamide.

24. The electrophoresis gel plate of claim 19, wherein the isoelectric focusing gel matrix comprises a pH value of between 2 and 12.

25-32. (canceled)

26. The electrophoresis gel plate of claim 1, wherein the polymerised gel matrix is suitable for use in a multi-compartment electrolyser apparatus.

27-32. (canceled)

33. The electrophoresis gel plate of claim 1, wherein the gel matrix is about 10% (v/v) thickness of the thickness of the microporous substrate.

34-39. (canceled)

40. The electrophoresis gel plate of claim 1, wherein the gel matrix is a monolayer.

41. The electrophoresis gel plate of claim 1, wherein the hydrophilic microporous membrane is constructed of a polymeric material.

42. (canceled)

43. The electrophoresis gel plate of claim 41, wherein the microporous membrane comprises a cellulosic material.

44-45. (canceled)

46. The electrophoresis gel plate of claim 1, wherein the microporous membrane comprises a porous substrate, and an insoluble cross-linked hydrophilic material.

47-49. (canceled)

50. The electrophoresis gel plate of claim 46, wherein the porous substrate comprises a polymer selected from the group consisting of poly(tetrafluoroethylene), polyvinylidene fluoride (PVDF), polyethylene, ultra-high molecular weight polyethylene (UPE), polysulfone, polyethersulfone, polypropylene, polymethylpentene, polyethylene terephthalate, polybutylene terephthalate, polyvinyl chloride and polyacrylonitriles.

51-61. (canceled)

62. A method of making the electrophoresis gel plate of claim 1, the method comprising wetting the hydrophilic microporous substrate with a casting solution, and treating the casting solution to effect polymerization.

63-73. (canceled)

74. A method of analyzing or separating macromolecules in a mixture comprising: (i) placing the mixture of macromolecules in a separator apparatus comprising at least one electrophoresis gel plate and (ii) performing electrophoresis on the mixture wherein the electrophoresis gel plate comprises a polymerized gel matrix supported by a hydrophilic microporous membrane.

75-79. (canceled)

80. A kit for analyzing or separating macromolecules in a mixture, the kit comprising one or more electrophoresis gel plates of claim 1, and buffers.

81. The kit of claim 80, wherein the kit further comprises at least one component selected from the group consisting of urea, thiourea, CHAPS and carrier ampholytes.

82-83. (canceled)

84. The kit of claim 80, wherein the kit further comprises a multicompartment electrolyser apparatus.

85. The kit of claim 80, wherein the kit further comprises instructions for use.
